(19) United States
(12) Patent Application Publication
(10) Pub. No.: US 2024/0290447 A1
(43) Pub. Date: Aug. 29, 2024

(54) PATIENT INFORMATION PROCESSING METHOD, PATIENT INFORMATION PROCESSING DEVICE, AND PATIENT INFORMATION PROCESSING SYSTEM

(71) Applicant: NIHON KOHDEN CORPORATION, Tokyo (JP)

(72) Inventors: Itaru Fukuhara, Tokorozawa-shi (JP); Mitsuhiro Oura, Tokorozawa-shi (JP); Syuhei Kawamura, Tokorozawa-shi (JP)

(73) Assignee: NIHON KOHDEN CORPORATION, Tokyo (JP)

(21) Appl. No.: 18/587,417

(22) Filed: Feb. 26, 2024

(30) Foreign Application Priority Data
Feb. 27, 2023 (JP) 2023-028465

(51) Int. Cl. G16H 10/60; G16H 50/30

(52) U.S. Cl. CPC G16H 10/60; G16H 50/30

(57) ABSTRACT

A patient information processing method is executed by one or more processors, and the method includes obtaining a plurality of pieces of vital data of a patient, calculating a vital score indicating a condition of the patient using at least the plurality of pieces of vital data, and generating summary information related to the vital score and/or the plurality of pieces of vital data during a specific period.

### CROSS-REFERENCE TO RELATED APPLICATIONS

The present application claims the benefit of priority of Japanese Patent Application No. 2023-028465, filed on Feb. 27, 2023, the content of which is incorporated herein by reference.

### TECHNICAL FIELD

The present disclosure relates to a patient information processing method, a patient information processing device, and a patient information processing system. Furthermore, the present disclosure relates to a program that causes a computer to execute the patient information processing method, and a computer readable storage medium that stores the program.

### BACKGROUND ART

In the world, a pandemic due to a novel coronavirus infectious disease (COVID-19) still rages, and in a specific medical institution, the management of a severe patient has become a very important factor with an explosive increase of a patient infected with the novel coronavirus. In particular, in a medical field, a vital score (for example, National Early Warning Score (NEWS)) indicating a condition of a patient is effectively used for the management of a severe patient.

JP2019-170851A discloses a patient information processing system that calculates a vital score for each patient based on vital data of the patient and displays a patient information list screen including a plurality of pieces of patient information. A medical worker can grasp the condition of each patient by checking each piece of patient information displayed on the patient information list screen.

### SUMMARY OF INVENTION

On the other hand, in the patient information list screen or the like disclosed in JP2019-170851A, it is very troublesome for the medical worker to grasp an overview (for example, a worst value or the like in an absence period) related to the condition of each patient in an absence period of the medical worker. For example, although vital data and a vital score of each patient in each time zone during the absence period can be manually checked, the medical worker needs to check the vital data and the vital score of each patient for each time zone during the absence period on a display screen, and then grasp the condition of each patient during the absence period. As described above, there is room for improvement in a user interface that displays the patient information since it is not easy to get the overview of the condition of each patient during the absence period.

An object of the present disclosure is to provide a patient information processing method, a patient information processing device, and a patient information processing system capable of easily grasping an overview related to a condition of a patient in a specific period such as an absence period of a medical worker. Further, another object of the present disclosure is to provide a program that causes a computer to execute the patient information processing method, and a computer readable storage medium that stores the program.

A patient information processing method according to an aspect of the presently disclosed subject matter is executed by one or more processors, and the method includes
obtaining a plurality of pieces of vital data of a patient,
calculating a vital score indicating a condition of the patient using at least the plurality of pieces of vital data, and
generating summary information related to the vital score and/or the plurality of pieces of vital data during a specific period.

According to the above method, the summary information related to the vital score and/or the plurality of pieces of vital data in the specific period is generated. As described above, a medical worker can easily grasp an overview of the condition of the patient in an absence period of the medical worker through the summary information.

A program that causes a computer to execute the patient information processing method and a computer readable medium that stores the program may be provided.

A patient information processing device according to an aspect of the presently disclosed subject matter includes
one or more processors, and
one or more memories configured to store a computer readable instruction.
When the computer readable instruction is executed by the one or more processors, the patient information processing device
   obtains a plurality of pieces of vital data of a patient,
   calculates a vital score indicating a condition of the patient using at least the plurality of pieces of vital data, and
   generates summary information related to the vital score and/or the plurality of pieces of vital data during a specific period.

According to the above configuration, it is possible to provide the patient information processing device capable of easily grasping the overview of the condition of the patient in the specific period such as an absence period of the medical worker. Thus, usability of the patient information processing device can be improved.

A patient information processing system according to an aspect of the presently disclosed subject matter includes
a server, and
a display terminal communicably connected to the server.
The patient information processing system
   obtains a plurality of pieces of vital data of a patient,
   calculates a vital score indicating a condition of the patient using at least the plurality of pieces of vital data,
   generates summary information related to the vital score and/or the plurality of pieces of vital data during a specific period, and
   displays the summary information.

According to the above configuration, it is possible to provide the patient information processing system capable of easily grasping the overview of the condition of the patient in the specific period such as an absence period of the medical worker. Thus, usability of the patient information processing system can be improved.

According to the present disclosure, it is possible to provide a patient information processing method, a patient information processing device, and a patient information processing system capable of easily grasping an overview related to a condition of a patient in a specific period such as an absence period of a medical worker. According to the present disclosure, it is possible to provide a program that causes a computer to execute the patient information processing method, and a computer readable storage medium that stores the program.

### BRIEF DESCRIPTION OF DRAWINGS

FIG. 1 is a diagram illustrating an example of a configuration of a patient information processing system according to an embodiment of the present disclosure (hereinafter, referred to as the present embodiment).

FIG. 2 is a diagram illustrating an example of a hardware configuration of a server.

FIG. 3 is a diagram illustrating an example of various tables in an in-hospital information database.

FIG. 4A is a diagram illustrating an example of an information table of a patient A.

FIG. 4B is a diagram illustrating an example of an information table of a patient B.

FIG. 4C is a diagram illustrating an example of an information table of a patient C.

FIG. 4D is a diagram illustrating an example of an information table of a patient D.

FIG. 5 is a diagram illustrating an example of a method of calculating a NEWS score.

FIG. 6 is a diagram illustrating an example of a medical worker information table.

FIG. 7 is a diagram illustrating an example of a hardware configuration of a display terminal.

FIG. 8 is a sequence diagram illustrating a patient information processing method according to the present embodiment.

FIG. 9 is a diagram illustrating an example of a patient information list screen on which each piece of patient information is displayed.

FIG. 10 is a diagram illustrating an example of the patient information list screen on which each piece of patient information and summary information are displayed.

### DESCRIPTION OF EMBODIMENTS

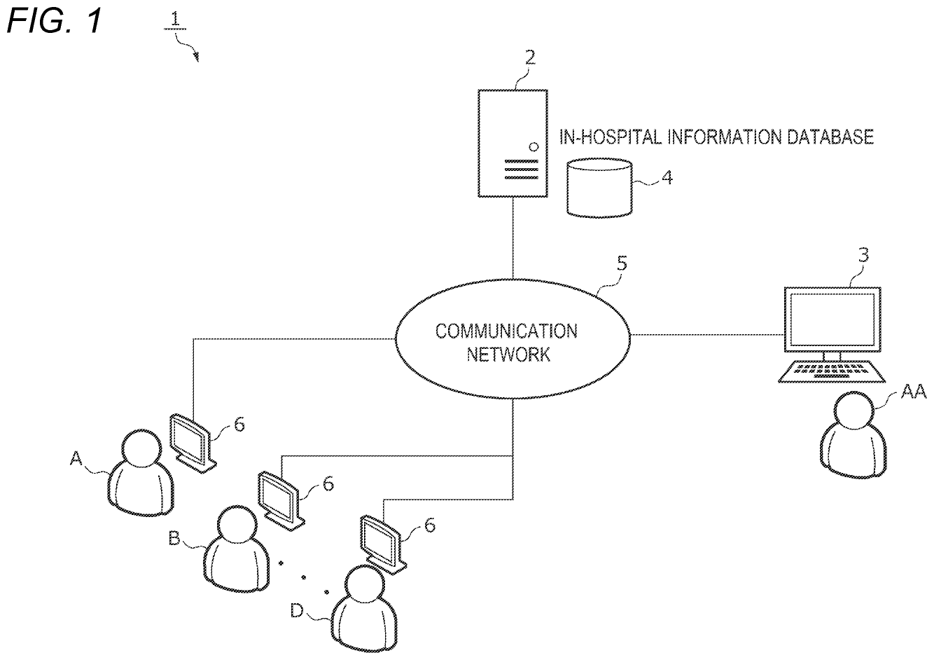

Hereinafter, the present embodiment will be described with reference to the drawings. First, a patient information processing system 1 according to the present embodiment will be described below with reference to FIG. 1. FIG. 1 is a diagram illustrating an example of a configuration of the patient information processing system 1 (hereinafter, referred to as the processing system 1) according to the present embodiment. As illustrated in FIG. 1, the processing system 1 includes a server 2, a plurality of bed-side monitors 6, and a display terminal 3. The server 2 is communicably connected to the plurality of bed-side monitors 6 and the display terminal 3 via a communication network 5.

The communication network 5 may be an in-hospital network including, for example, a local area network (LAN) or a wide area network (WAN). Further, the communication network 5 may include the in-hospital network and the Internet. In this case, the server 2 may be a server installed in the in-hospital network or an external server (for example, a cloud server) installed outside the in-hospital network.

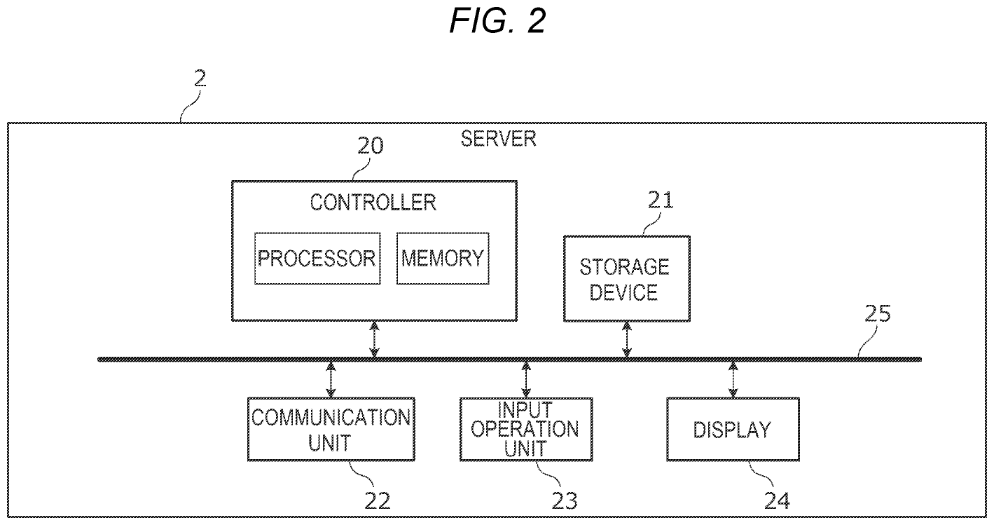

The server 2 includes an in-hospital information database 4. The in-hospital information database 4 is built in, for example, a storage device 21 (see FIG. 2). The in-hospital information database 4 may be built in another database server communicably connected to the server 2.

Next, a hardware configuration of the server 2 will be described with reference to FIG. 2. FIG. 2 is a diagram illustrating an example of the hardware configuration of the server 2.

As illustrated in FIG. 2, the server 2 includes a controller 20, the storage device 21, a communication unit 22, an input operation unit 23, and a display 24. These components are communicably connected to one another via a bus 25.

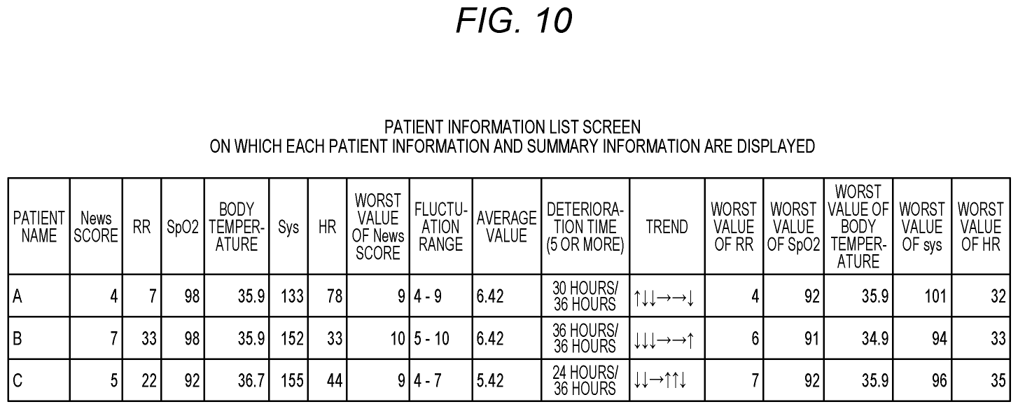

The controller 20 includes one or more memories and one or more processors. Each memory is configured to store a computer readable instruction (program). For example, the memory may include a read only memory (ROM) in which various programs are stored, and a random access memory (RAM) having a plurality of work areas in which various programs executed by the one or more processors are stored. Each processor may include at least one of, for example, a central processing unit (CPU), a micro processing unit (MPU), and a graphics processing unit (GPU). The CPU may include a plurality of CPU cores. The GPU may include a plurality of GPU cores. The processor may be configured to load a program designated from various programs incorporated in the storage device 21 or ROM into RAM, and configured to execute various processes in cooperation with the RAM. In particular, the processor loads a patient information processing program that executes a series of processes (for example, see FIG. 10) of the server 2 into the RAM, and executes the program in cooperation with the RAM, whereby the controller 20 executes the series of processes illustrated in FIG. 10. Details of the patient information processing program will be described later.

The storage device 21 is a storage device (storage) such as a hard disk drive (HDD), a solid state drive (SSD), or a flash memory, and is configured to store programs and various data. The patient information processing program may be incorporated in the storage device 21. In addition, the in-hospital information database 4 is stored in the storage device 21.

The communication unit 22 is configured to connect the server 2 to the communication network 5. Specifically, the communication unit 22 may include various wired connection terminals for communicating with each device disposed on the communication network 5. In addition, the communication unit 22 may include a wireless communication module for wirelessly communicating with each device. The input operation unit 23 is, for example, a touch panel, a mouse, and/or a keyboard, which are overlaid and disposed over the display 24. The input operation unit 23 is configured to receive an input operation of an operator and generate an operation signal corresponding to the input operation of the operator. The display 24 includes, for example, a liquid crystal panel or an organic EL panel.

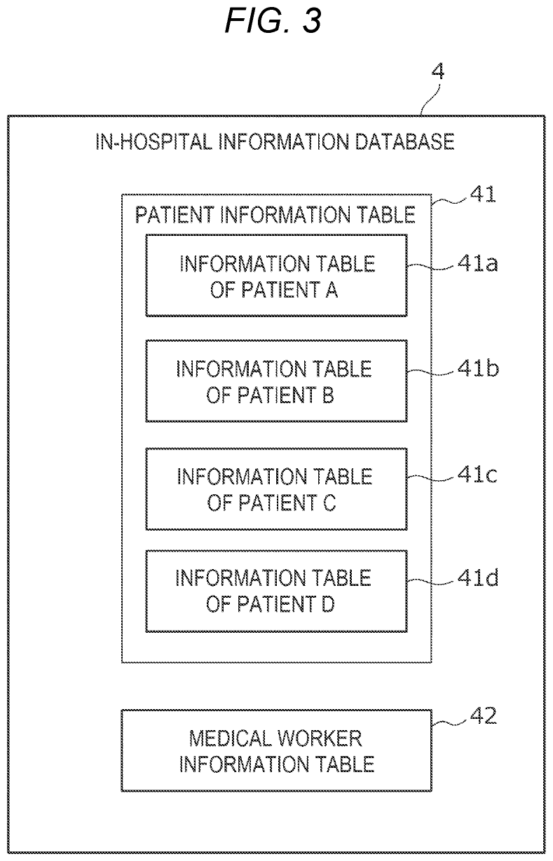

Next, the in-hospital information database 4 built in the storage device 21 will be described with reference to FIG. 3. As illustrated in FIG. 3, the in-hospital information database 4 includes a patient information table 41 and a medical worker information table 42. The patient information table 41 includes an information table 41a of a patient A, an information table 41b of a patient B, an information table 41c of a patient C, and an information table 41d of a patient D. In the present embodiment, information tables of four patients are stored in the in-hospital information database 4 for simplification of description, but information tables of thousands of patients or more may be stored in the in-hospital information database 4.

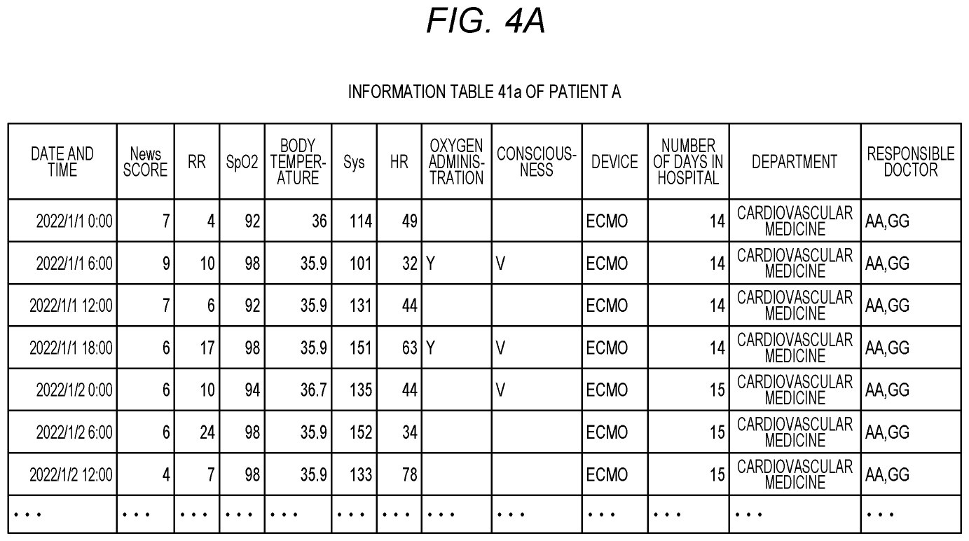

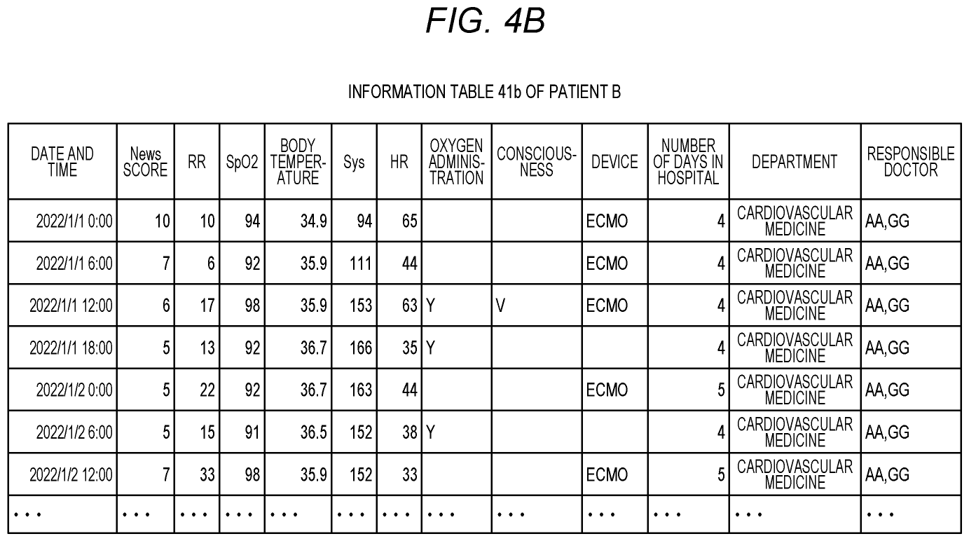

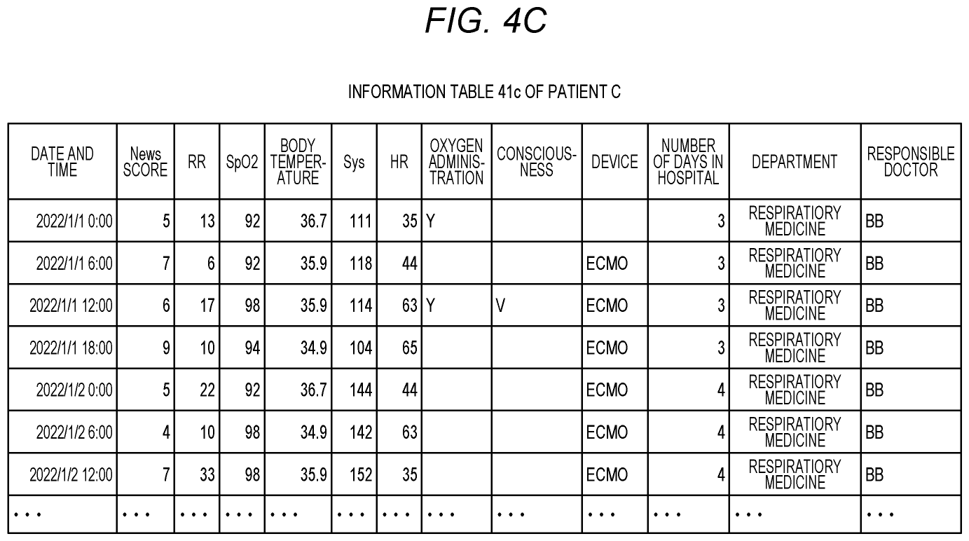

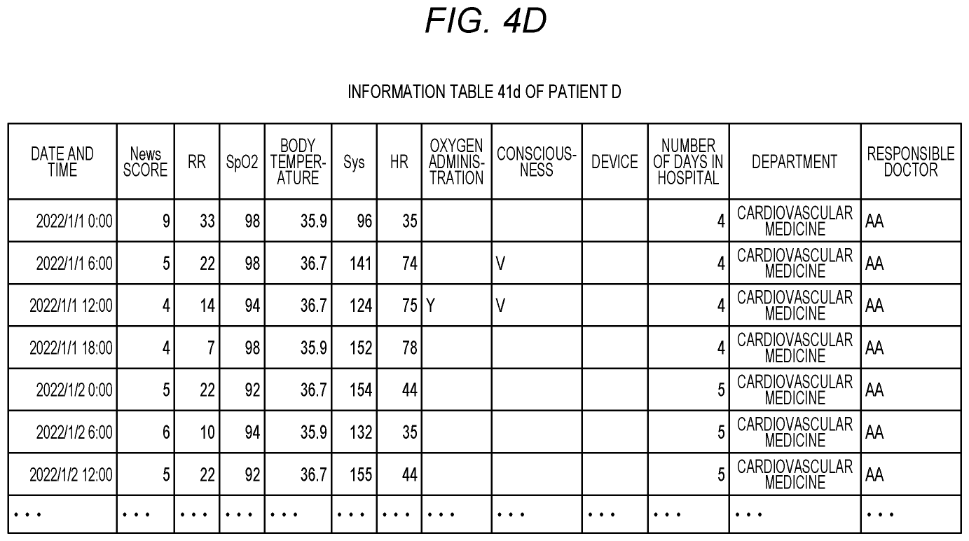

FIG. 4A is a diagram illustrating an example of the information table 41a of the patient A. As illustrated in FIG. 4A, the information table 41a of the patient A (hereinafter, the information table 41a) includes information indicating vital data of the patient A, information indicating a NEWS score of the patient A, information indicating a medical device connected to the patient A, and information indicating the number of days in a hospital of the patient A. Furthermore, the information table 41a includes information indicating a department where the patient A receives a medical examination and information indicating a responsible doctor in charge of the patient A. The server 2 obtains the information in the information table 41a at predetermined time intervals (six hours in this example) and stores the information in the information table 41a. That is, information related to the patient A in each time zone is stored in the information table 41a. A time interval at which each piece of information in the information table 41a is obtained is not particularly limited.

FIG. 4B is a diagram illustrating an example of the information table 41b of the patient B. As illustrated in FIG. 4B, the information table 41b of the patient B (hereinafter, the information table 41b) includes information indicating vital data of the patient B, information indicating a NEWS score of the patient B, information indicating a medical device connected to the patient B, and information indicating the number of days in a hospital of the patient B. Furthermore, the information table 41b includes information indicating a department where the patient B receives a medical examination and information indicating a responsible doctor in charge of the patient B. Information related to the patient B in each time zone is stored in the information table 41b. FIG. 4C illustrates an example of the information table 41c of the patient C (hereinafter, referred to as the information table 41c). Information related to the patient C in each time zone is stored in the information table 41c. FIG. 4D illustrates an example of the information table 41d of the patient D (hereinafter, referred to as the information table 41d). Information related to the patient D in each time zone is stored in the information table 41d.

As illustrated in FIGS. 4A to 4D, information indicating vital data of a patient includes information indicating a respiration rate (RR), an oxygen saturation (SpO2), a body temperature, a systolic blood pressure (Sys), and a heart rate (HR).

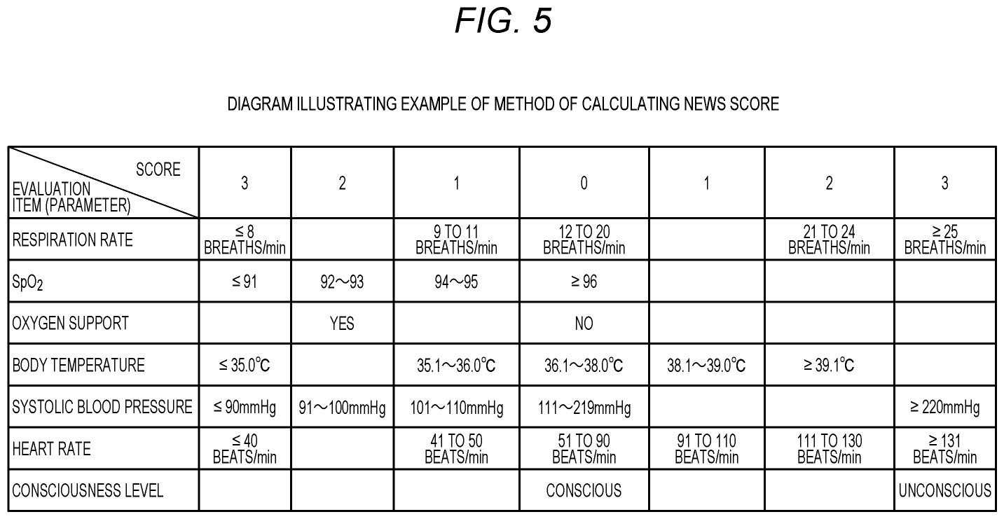

In the description of the present embodiment, a NEWS score is calculated as an example of a vital score indicating a condition of the patient. For example, as illustrated in FIG. 5, the server 2 calculates the NEWS score based on each piece of vital data, the presence or absence of oxygen administration, and the presence or absence of consciousness of the patient. Specifically, the server 2 calculates a sub score of each of the plurality of pieces of vital data in accordance with a comparison between each of the plurality of pieces of vital data and a reference range set for each of the plurality of pieces of vital data. For example, when the respiration rate (RR) is 12 breaths/min to 20 breaths/min, a sub score related to the respiration rate is zero. When the respiration rate is 9 breaths/min to 11 breaths/min, the sub score related to the respiration rate is one. When the respiration rate is 21 breaths/min to 24 breaths/min, the sub score related to the respiration rate is two. When the respiration rate is 8 breaths/min or less or 25 breaths/min or more, the sub score related to the respiration rate is three. Furthermore, the server 2 calculates a sub score related to the oxygen administration and a sub score related to the consciousness of the patient. For example, when the oxygen administration is performed, the sub score related to the oxygen administration is two. On the other hand, when the oxygen administration is not performed, the sub score related to the oxygen administration is zero. When the patient is not conscious, the sub score related to the consciousness of the patient is three. On the other hand, when the patient is conscious, the sub score related to the consciousness of the patient is zero.

In this manner, the server 2 calculates the NEWS score of the patient by summing up the calculated sub scores. For example, as illustrated in FIG. 4A, a method of calculating the NEWS score of the patient A at a time point of 0:00 on Jan. 1, 2022 will be described. In this case, since a respiration rate (RR) of the patient A is 4 breaths/min, the sub score related to the respiration rate is three. The oxygen saturation (SpO2) of the patient A is 92%, and therefore, a sub score related to the oxygen saturation is two. A body temperature of the patient A is 36° C., and therefore, a sub score related to the body temperature is one. Systolic blood pressure (Sys) of the patient A is 114 mmHg, and therefore, a sub score related to the systolic blood pressure is zero. A heart rate (HR) of the patient A is 49 beats/min, and therefore, a sub score related to the heart rate is one. Since the patient A is not administered with oxygen, the sub score related to the oxygen administration is zero. Since the patient A is conscious, the sub score related to the consciousness of the patient is zero. As described above, the NEWS score of the patient A at the time point of 0:00 on Jan. 1, 2022 is 7 (=3+2+1+1) by summing up the sub scores.

In the present embodiment, the vital score is not limited to the NEWS score. For example, as another example of the vital score, SOFA, qSOFA, APACHE2, BSAS, NIHSS, or the like may be adopted. Further, in the present embodiment, the respiration rate, the oxygen saturation, the body temperature, the systolic blood pressure, and the heart rate are obtained as the vital data of the patient in order to calculate the NEWS score, but a type of the vital data of the patient may be changed in accordance with a type of the vital score to be adopted.

The vital data in the information tables 41a to 41d are obtained at predetermined time intervals (six hours in this example). As illustrated in FIG. 1, the vital data of the patients A to D is transmitted from the plurality of bed-side monitors 6 (an example of a patient monitor) to the server 2 via the communication network 5 at predetermined time intervals. Each of the plurality of bed-side monitors 6 is connected to respective one of the patients A to D. Each of the bed-side monitors 6 includes a plurality of vital sensors that measure the vital data such as a respiration rate and a heart rate of the patient. The plurality of vital sensors may include, for example, a respiration sensor, an SpO2 sensor, a body temperature sensor, a blood pressure sensor, and an electrocardiogram sensor.

When an extracorporeal membrane oxygenation (ECMO) is connected to the patient, information indicating ECMO is input as information indicating a medical device connected to the patient. When a respirator is connected to the patient, information indicating the respirator is input as information indicating the medical device connected to the patient. The medical device connected to the patient is not limited to ECMO or the respirator, and may be a medical device such as a CO2 sensor or a pacemaker.

Information indicating the number of days in a hospital of the patient may be calculated by the server 2 based on a difference between a hospitalization date of the patient and a current date. In this manner, the information indicating the number of days in a hospital of the patient is updated at predetermined time intervals. Each of the information indicating the presence or absence of oxygen administration, the information indicating the presence or absence of the consciousness of the patient, the information indicating the medical device connected to the patient, and the information indicating the hospitalization date of the patient may be directly input to the server 2 through the input operation unit 23 of the server 2. Alternatively, the server 2 may receive the information from an electronic medical record server (not illustrated) disposed on the communication network 5 at predetermined time intervals.

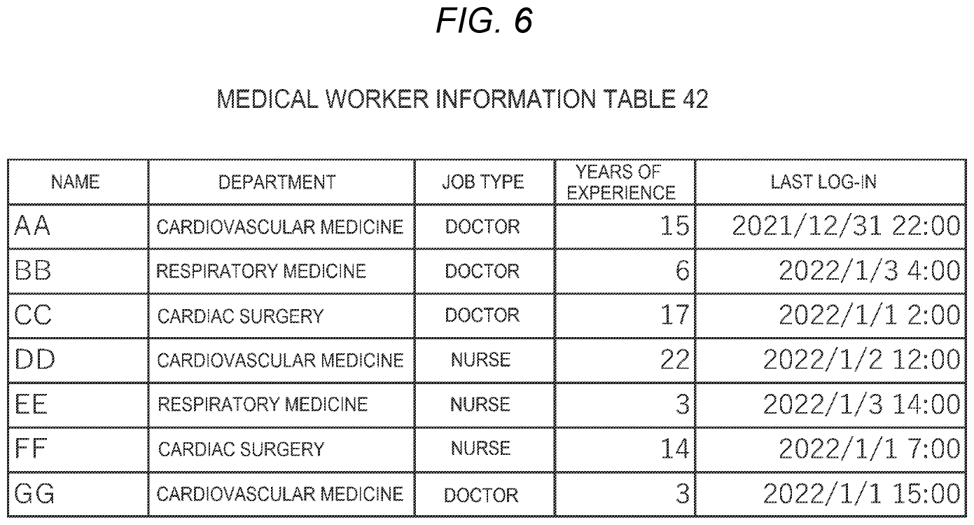

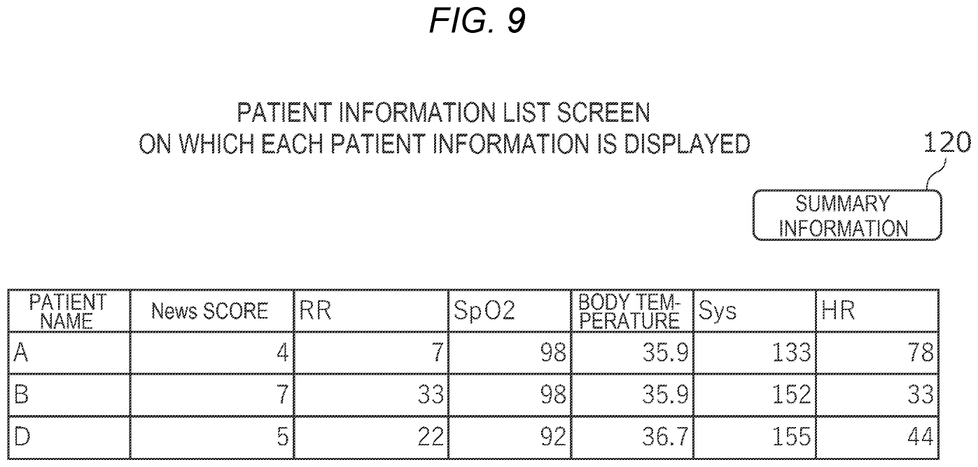

FIG. 6 is a diagram illustrating an example of the medical worker information table 42. As illustrated in FIG. 6, the medical worker information table 42 includes information indicating a name of the medical worker, a department to which the medical worker belongs, a job type, years of experience, and a last log-in date-and-time. Here, information indicating the last log-in date-and-time is information indicating a last date and time when the medical worker logs in a WEB application that displays a patient information list screen (see FIG. 9) including a plurality of pieces of patient information. The server 2 may update the information indicating the last log-in date-and-time in the medical worker information table 42 at an arbitrary timing. For example, the server 2 may update information related to a last log-in date-and-time of a medical worker AA when receiving, from the display terminal 3, a signal indicating log-out of the medical worker AA for the WEB application.

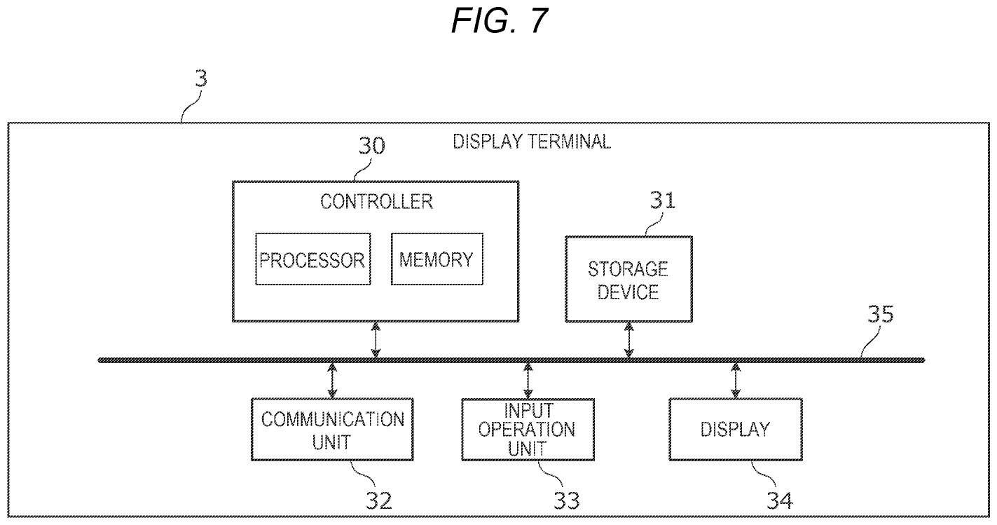

Next, a hardware configuration of the display terminal 3 will be described below with reference to FIG. 7. FIG. 7 is a diagram illustrating an example of the hardware configuration of the display terminal 3. As illustrated in FIG. 7, the display terminal 3 includes a controller 30, a storage device 31, a communication unit 32, an input operation unit 33, and a display 34. These components are communicably connected to one another via a bus 35. A type of the display terminal 3 is not particularly limited as long as the display terminal 3 is a display terminal managed by the medical worker and has a function (for example, a WEB browser) capable of displaying the patient information list screen (see FIG. 9 and the like) to be described later. The display terminal 3 may be a patient monitor, a personal computer, a workstation, a smartphone, a tablet, or a wearable device (for example, an AR glass or a head-mounted display) worn on a body (for example, an arm or a head) of a medical worker.

The controller 30 includes one or more memories and one or more processors. Each memory is configured to store a computer readable instruction (program). For example, the memory includes a ROM and a RAM. Each processor includes at least one of a CPU, an MPU, and a GPU, for example. The processor may be configured to load a program designated from various programs incorporated in the storage device 31 or ROM into RAM, and configured to execute various processes in cooperation with the RAM. The storage device 31 is a storage device such as an HDD, an SSD, or a flash memory, and is configured to store programs and various data.

The communication unit 32 is configured to connect the display terminal 3 to the communication network 5. Specifically, the communication unit 32 may include various wired connection terminals for communicating with each device disposed on the communication network 5. In addition, the communication unit 32 may include a wireless communication module for wirelessly communicating with each device. The input operation unit 33 is, for example, a touch panel, a mouse, and/or a keyboard, which are overlaid and disposed over the display 34. For example, in response to an input operation performed by the medical worker AA on the input operation unit 33, an input operation signal indicating the input operation performed by the medical worker AA is transmitted from the input operation unit 33 to the controller 30. Thereafter, the controller 30 executes a predetermined process in response to the received input operation signal. The display 34 includes, for example, a liquid crystal panel or an organic EL panel. The patient information list screen (see FIG. 9) to be described later is displayed on a display screen of the display 34. When the display terminal 3 is a head-mounted display, the patient information list screen may be disposed in a virtual space (Metaverse).

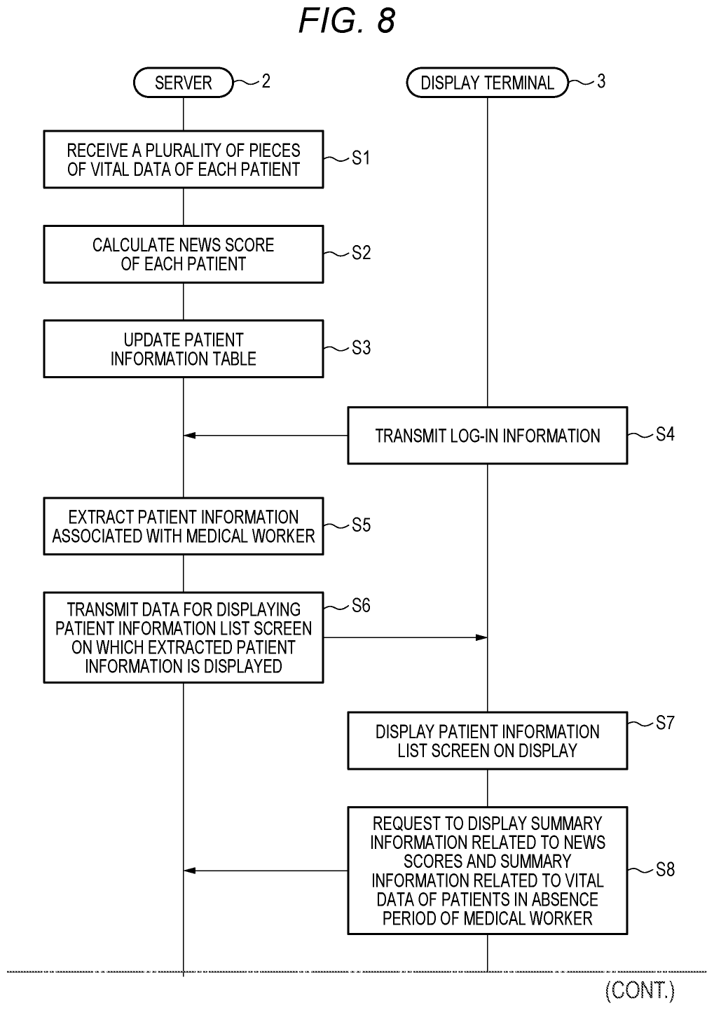

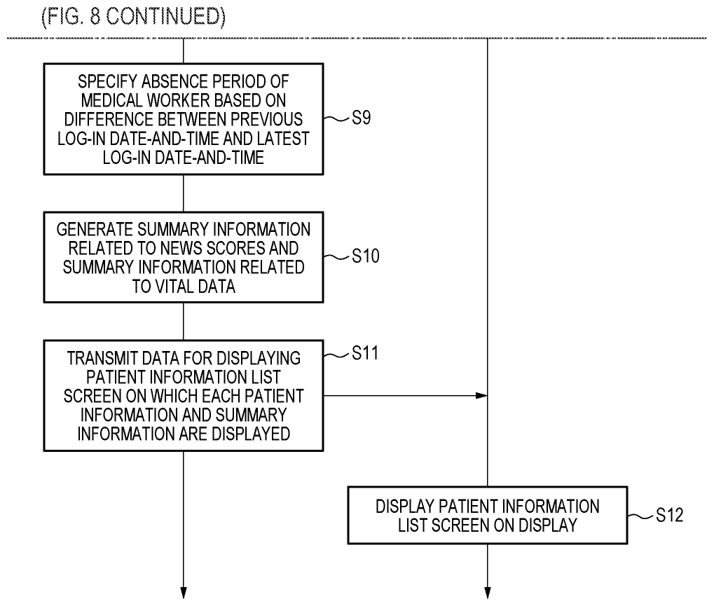

Next, a patient information processing method according to the present embodiment will be described below with reference to FIG. 8. FIG. 8 is a sequence diagram illustrating the patient information processing method according to the present embodiment. As illustrated in FIG. 8, the patient information processing method according to the present embodiment is executed by cooperation between the server 2 and the display terminal 3. In addition, in the following description, it is assumed that the medical worker AA accesses the server 2 through the display terminal 3.

In step S1, the server 2 obtains a plurality of pieces of vital data (respiration rate, oxygen saturation, body temperature, systolic blood pressure, and heart rate) of each of the patients A to D from the plurality of bed-side monitors 6 connected to the patients A to D. As described above, the server 2 may obtain the vital data of the patients A to D from the bed-side monitor 6 via the communication network 5 at predetermined time intervals.

In step S2, the server 2 calculates a NEWS score of each of the patients A to D based on the plurality of vital data of each of the patients A to D, the presence or absence of the oxygen administration of each of the patients A to D, and the presence or absence of the consciousness of each of the patients A to D. As described above, the server 2 can calculate the NEWS score of the patient by summing a sub score of each piece of vital data, the sub score related to the oxygen administration, and the sub score related to the presence or absence of the consciousness.

In step S3, the server 2 updates the information tables 41a to 41d (see FIGS. 4A to 4D) of the patients A to D. In particular, the server 2 updates the vital data of the patient, the NEWS score of the patient, the medical device, the number of days in a hospital of the patient, the presence or absence of the oxygen administration, the presence or absence of the consciousness of the patient, the department, and the information on the responsible doctor.

Next, in step S4, the display terminal 3 transmits log-in information of the medical worker AA for accessing the server 2 to the server 2 via the communication network 5. Specifically, when the medical worker AA inputs a log-in ID and a password of the medical worker AA on the WEB browser displayed on the display terminal 3, the display terminal 3 transmits the log-in information indicating the log-in ID and the password of the medical worker AA to the server 2. Authentication of the medical worker AA is not limited to log-in authentication.

In step S5, the server 2 authenticates the log-in information of the medical worker AA, and then extracts patient information associated with the medical worker AA. Specifically, the server 2 extracts the patient information associated with the medical worker AA by referring to the information on the responsible doctor in each of the information tables 41a to 41d (see FIGS. 4A to 4D). In this example, since patients treated by the medical worker AA are the patients A, B, and D, patient information of the patients A, B, and D is extracted as the patient information associated with the medical worker AA. Next, in step S6, the server 2 transmits, to the display terminal 3, data for displaying the patient information list screen on which the patient information of the patients A, B, and D is displayed. Here, the data for displaying the patient information list screen may be a code (HTML, CSS, JavaScript, JSON data (or XML data), or the like) for displaying the patient information list screen on the WEB browser of the display terminal 3, or may be image data indicating the patient information list screen.

In step S7, the display terminal 3 displays the patient information list screen on the display screen of the display 34 based on the data for displaying the patient information list screen, which is received from the server 2. For example, the WEB browser installed in the display terminal 3 may render the patient information list screen by interpreting the data (HTML, CSS, JavaScript, JSON data (or XML data), or the like) for displaying the patient information list screen. As illustrated in FIG. 9, the patient information list screen including the patient information of the patients A, B, and D is displayed on the display 34 of the display terminal 3. Here, each piece of patient information displayed on the patient information list screen includes a plurality of pieces of vital data (respiration rate, oxygen saturation, body temperature, systolic blood pressure, and heart rate) and a NEWS score.

In step S8, the display terminal 3 requests the server 2 to display summary information related to NEWS scores and summary information related to the vital data of the patients A, B, and D in an absence period (an example of a specific period) of the medical worker AA in response to the input operation of the medical worker AA. In this regard, as illustrated in FIG. 9, when the medical worker AA clicks an input button 120 for displaying the summary information, a signal for requesting the display of the summary information may be transmitted from the display terminal 3 to the server 2. The summary information related to the NEWS score in the absence period is information indicating an overview of the NEWS score in the absence period. More specifically, the summary information is information indicating at least one of a worst value, a fluctuation range, an average value, a mode value, a median value, a deterioration time, and a trend of the NEWS score in the absence period.

Here, the worst value of the NEWS score in the absence period is a maximum value of the NEWS score in the absence period. The fluctuation range of the NEWS score in the absence period indicates a range between the maximum value and a minimum value of the NEWS score in the absence period. The deterioration time of the NEWS score indicates a period in which a value of the NEWS score is deteriorated. The trend of the NEWS score indicates an ascending trend, a descending trend, or a lateral trend of the NEWS score in each time zone.

The summary information related to the vital data in the absence period is information indicating an outline of the vital data (respiration rate, oxygen saturation, body temperature, systolic blood pressure, and heart rate) in the absence period. More specifically, the summary information is information indicating at least one of a worst value, a fluctuation range, an average value, a mode value, a median value, a deterioration time, and a trend of each vital data in the absence period.

Here, the worst value of the vital data in the absence period is a maximum value or a minimum value of the vital data in the absence period. The fluctuation range of the vital data in the absence period indicates a range between the maximum value and the minimum value of the vital data in the absence period. The deterioration time of the vital data indicates a period in which a value of the vital data is deteriorated. The trend of the vital data indicates an ascending trend, a descending trend, or a lateral trend of the vital data in each time zone.

Next, when the server 2 receives the signal requesting the display of the summary information from the display terminal 3, the server 2 specifies the absence period of the medical worker AA based on a difference between a previous log-in date-and-time and a latest log-in date-and-time of the medical worker AA for the WEB application displaying the patient information list screen (step S9). In this regard, the server 2 may specify the previous log-in date-and-time (last log-in date-and-time) of the medical worker AA by referring to the medical worker information table 42 illustrated in FIG. 6, and then specify the absence period of the medical worker AA based on the difference between the previous log-in date-and-time and the latest log-in date-and-time of the medical worker AA. For example, when the previous log-in date-and-time of the medical worker AA is 22:00 on Dec. 31, 2021 and a current log-in date-and-time is 15:00 on Jan. 2, 2022, the server 2 may specify a period from 22:00 on Dec. 31, 2021 to 15:00 on Jan. 2, 2022 as the absence period of the medical worker AA.

In step S10, the server 2 generates the summary information related to the NEWS scores of the patients A, B, and D in the absence period of the medical worker AA, and generates the summary information related to the vital data of the patients A, B, and D in the absence period. As described above, the server 2 may generate the information indicating at least one of the worst value, the fluctuation range, the average value, the mode value, the median value, the deterioration time, and the trend of the NEWS score in the absence period as the summary information of the NEWS score. The server 2 may generate the information indicating at least one of the worst value, the fluctuation range, the average value, the mode value, the median value, the deterioration time, and the trend of the vital data in the absence period as the summary information of the vital data.

FIG. 10 illustrates an example of the patient information list screen on which each piece of patient information and the summary information of the NEWS score and the vital data are displayed. In the example illustrated in FIG. 10, the worst value, the fluctuation range, the average value, the deterioration time, and the trend of the NEWS score of each patient are displayed on the patient information list screen as the summary information of the NEWS score of each patient. Here, the deterioration time of the NEWS score indicates a time during which the NEWS score is deteriorated in the absence period (in this example, 36 hours). The trend of the NEWS score indicates a trend (ascending, descending, or lateral) of the NEWS score at predetermined time intervals. As the summary information of the NEWS score, at least one of the worst value, the fluctuation range, the average value, the mode value, the median value, the deterioration time, and the trend of the NEWS score may be displayed on the patient information list screen.

In the example illustrated in FIG. 10, worst values of the plurality of vital data of each patient are displayed on the patient information list screen as the summary information of the plurality of vital data (respiration rate, oxygen saturation, body temperature, systolic blood pressure, and heart rate) of each patient in the absence period. As the summary information of the plurality of vital data, at least one of the worst value, the fluctuation range, the average value, the mode value, the median value, the deterioration time, and the trend of each of the plurality of vital data may be displayed on the patient information list screen.

Returning to FIG. 8, in step S11, the server 2 transmits, to the display terminal 3, the data for displaying the patient information list screen on which the patient information and the summary information of the patients A, B, and D are displayed. As described above, the data for displaying the patient information list screen may be a code for displaying the patient information list screen on the WEB browser of the display terminal 3, or may be image data indicating the patient information list screen.

In step S12, the display terminal 3 displays the patient information list screen on the display screen of the display 34 based on the data for displaying the patient information list screen, which is received from the server 2. As illustrated in FIG. 10, the patient information list screen including the patient information and the summary information of the patients A, B, and D is displayed on the display 34 of the display terminal 3. Here, the patient information of the patients A, B, and D includes information indicating current NEWS scores of the patients A, B, and D and information indicating current vital data of the patients A, B, and D. As described above, the information indicating the current NEWS scores, the information indicating the current vital data, and the summary information of the NEWS score and the vital data are displayed on the same screen.

According to the present embodiment, the medical worker AA can easily grasp an overview of the condition of each patient during the absence period through the summary information displayed on the patient information list screen. That is, because of the summary information, it is not necessary for the medical worker AA to grasp the overview of the condition of each patient during the absence period after checking, on the patient information list screen one by one by hand, the vital data and the NEWS score of each patient in each time zone during the absence period. As described above, it is possible to provide a user interface that makes it possible to easily grasp the overview related to the condition of each patient in the absence period of the medical worker, and usability of the WEB application that displays the patient information list screen is further improved.

According to the present embodiment, the absence period of the medical worker AA is automatically determined based on the difference between the previous log-in date-and-time and the latest log-in date-and-time of the medical worker AA for the WEB application. In this manner, the medical worker AA can easily grasp the overview of the condition of each patient in the absence period without performing an operation of inputting the absence period.

In the present embodiment, the absence period of the medical worker AA is automatically set on the server 2 side, but the absence period may be manually set by the medical worker AA. In this regard, an input region for inputting the absence period of the medical worker may be provided on the patient information list screen illustrated in FIG. 9. When the medical worker AA inputs information related to the absence period in the input region, information indicating the absence period designated by the medical worker AA is transmitted from the display terminal 3 to the server 2. Thereafter, the server 2 generates the summary information of the received NEWS score and the vital data in the absence period.

In the present embodiment, although the summary information of the NEWS score and the vital data is displayed on the patient information list screen, the summary information of either the NEWS score or the vital data may be displayed on the patient information list screen. In this case, the medical worker can easily grasp the overview of the condition of each patient during the absence period through the summary information of the NEWS score or the vital data displayed on the patient information list screen.

An order of processes illustrated in FIG. 8 is an example, and the order of the processes is not particularly limited. Although the patient information list screen is displayed on the display terminal 3 in the present embodiment, the patient information list screen may be displayed on the display 24 of the server 2. In this case, the medical worker AA operates the server 2 instead of the display terminal 3, whereby the patient information list screen is displayed on the display 24 of the server 2.

In order to implement the patient information processing method according to the present embodiment by software, a patient information processing program may be incorporated in advance in the storage device and/or the ROM of the server 2 and/or the display terminal 3. Alternatively, the patient information processing program may be stored in a computer readable storage medium such as a magnetic disk (for example, HDD and a floppy disk), an optical disk (for example, CD-ROM, DVD-ROM, and Blu-ray (registered trademark) disk), a magneto optical disk (for example, MO), or a flash memory (for example, an SD card, a USB memory, and SSD). In this case, the patient information processing program stored in the storage medium may be incorporated in the storage device. Further, after the program incorporated in the storage device is loaded onto RAM, one or more processors may execute the program loaded on RAM. In this manner, the patient information processing method according to the present embodiment is executed by the server 2 and/or the display terminal 3. In addition, the patient information processing program may be downloaded from an external computer on a communication network. In this case, the downloaded program may be also incorporated in the storage device.

Although the embodiment of the presently disclosed subject matter has been described above, the technical scope of the presently disclosed subject matter should not be construed as being limited to the description of the present embodiment. The present embodiment is merely an example, and it is understood by those skilled in the art that various modifications of the embodiment are possible within the scope of the disclosed subject matter described in the claims. The technical scope of the presently disclosed subject matter should be determined based on the scope of the disclosed subject matter described in the claims and equivalents thereof.

What is claimed is:

1. A patient information processing method executed by one or more processors, the method comprising:
obtaining a plurality of pieces of vital data of a patient;
calculating a vital score indicating a condition of the patient using at least the plurality of pieces of vital data; and
generating summary information related to the vital score and/or the plurality of pieces of vital data during a specific period.

2. The patient information processing method according to claim 1, further comprising:
a step of displaying the summary information.

3. The patient information processing method according to claim 2, wherein
the step of displaying the summary information includes a step of displaying, on the same screen, at least one of information indicating a plurality of pieces of current vital data and information indicating a current vital score, and the summary information.

4. The patient information processing method according to claim 1, further comprising:
a step of determining the specific period according to an input operation of a user.

5. The patient information processing method according to claim 1, further comprising:
a step of automatically determining the specific period based on a difference between a previous log-in date-and-time and a latest log-in date-and-time for an application that displays a plurality of pieces of patient information each of which is associated with respective one of a plurality of patients.

6. The patient information processing method according to claim 1, wherein
the summary information includes
summary information related to the vital score in the specific period, and
summary information related to the plurality of pieces of vital data in the specific period.

7. The patient information processing method according to claim 1, wherein
the summary information related to the vital score is information indicating at least one of a worst value, a fluctuation range, an average value, a mode value, a median value, and a trend of the vital score in the specific period.

8. The patient information processing method according to claim 1, wherein
the summary information related to the plurality of pieces of vital data is information indicating at least one of a worst value, a fluctuation range, an average value, a mode value, a median value, and a trend of each of the plurality of pieces of vital data in the specific period.

9. The patient information processing method according to claim 1, wherein
the step of obtaining the vital data includes a step of obtaining a plurality of pieces of vital data of each of a plurality of patients,
the step of calculating the vital score includes a step of calculating a vital score of each of the plurality of patients,
the patient information processing method further comprises a step of displaying a plurality of pieces of patient information each of which is associated with respective one of the plurality of patients, on a patient information list screen, and
each of the plurality of pieces of patient information includes the plurality of pieces of vital data and the vital score which are associated with the corresponding patient.

10. A computer readable medium that stores a program that causes a computer to execute the patient information processing method according to claim 1.

11. A patient information processing device, comprising:
one or more processors; and
one or more memories configured to store a computer readable instruction, wherein when the computer readable instruction is executed by the one or more processors, the patient information processing device
obtains a plurality of pieces of vital data of a patient,
calculates a vital score indicating a condition of the patient using at least the plurality of pieces of vital data, and
generates summary information related to the vital score and/or the plurality of pieces of vital data during a specific period.

12. A patient information processing system, comprising:
a server; and
a display terminal communicably connected to the server, wherein
the patient information processing system
obtains a plurality of pieces of vital data of a patient,
calculates a vital score indicating a condition of the patient using at least the plurality of pieces of vital data,
generates summary information related to the vital score and/or the plurality of pieces of vital data during a specific period, and
displays the summary information.
